(19) United States
(12) Patent Application Publication
(10) Pub. No.: US 2025/0248762 A1
(43) Pub. Date: Aug. 7, 2025

(54) METHOD, COMPUTING DEVICE, SYSTEM AND COMPUTER PROGRAM PRODUCT FOR ASSESSMENT OF INTERCONNECTION(S) BETWEEN ORTHOPEDIC IMPLANT(S) AND BONE TISSUE(S)

(71) Applicant: 25SEGMENTS AG, Zürich (CH)

(72) Inventors: Jonas WIDMER, Zürich (CH); Marie-Rosa FASSER, Zürich (CH); Mazda FARSHAD, Zumikon (CH)

(73) Assignee: 25SEGMENTS AG, CH-8008 Zürich (CH)

(21) Appl. No.: 18/852,651

(22) PCT Filed: Mar. 21, 2023
(86) PCT No.: PCT/EP2023/057197

§ 371 Date: Sep. 30, 2024

(30) Foreign Application Priority Data
Mar. 30, 2022 (CH) CH000359/2022

(51) Int. Cl. A61B 34/10; G06T 7/00; G06T 17/20

(52) U.S. Cl. CPC A61B 34/10; G06T 7/0012; G06T 17/20; A61B 2034/104; A61B 2034/105; G06T 2207/10081; G06T 2207/30008; G06T 2207/30052

(57) ABSTRACT

Computer implemented method, computing device, system and computer pro-gram product for assessment of interconnection(s) between orthopaedic implant(s) and a bone tissue(s) by: extracting location specific material properties of the bone tissue(s) from pre- and/or post-operative images; generating a 3D-Finite Element Model comprising first finite elements representing the orthopaedic implant interconnected to the bone tissue(s), and second finite elements representing at least part of the bone tissue(s) surrounding the orthopaedic implant; applying the location-specific material properties to the second finite elements; applying at least one external load to the one or more of the first finite elements; determining internal stresses of the 3D-Finite Element Model; and outputting an assessment of the interconnection between orthopaedic implant(s) and the bone tissue(s) based on the determining internal stresses.

### BACKGROUND OF THE INVENTION

### Field of the Invention

The present disclosure relates to a method, computing device, system and computer program product for assessment of interconnection(s) between orthopaedic implant(s) and bone tissue(s).

The present disclosure further relates to a computer implemented method, computing device, system and computer program product to support selection between different types of pedicle screws to be applied in a spine of a specific patient.

### Discussion of Related Art

Orthopaedic surgical interventions using orthopaedic implants are common procedures for the treatment of conditions involving the musculoskeletal system. In particular, spinal fixation of the spine is a gold standard surgical intervention and is employed for spine stabilization through the fusion of spinal segments.

However, loosening of orthopaedic implants, such as pedicle screw(s) after surgery is a common complication that can lead to complications such as persistent pain, failed fusion, and the need for revision surgery. For example, pedicle screw loosening has been reported to occur in about 10% of patients, and this percentage increases to above 60% for patients affected by osteoporosis. Hence there is a need for the assessment of the interconnection between orthopaedic implant(s) and the bone tissue. In particular, there is a need to predict the risk of pedicle screw loosening through the examination of subject-specific biomechanical aspects for the improvement of surgical outcomes.

There is no prior art quantitative and reliable measure for the assessment of the interconnection between orthopaedic implant(s) and the bone tissue. In particular, there is no prior art quantitative and reliable measure to identify level-specific risk of loosening of pedicle screws besides a general evaluation of bone tissue(s) quality from image data.

Finite element and musculoskeletal modelling are known methods in biomechanical research and can be complementary to each other. However, until now, the clinical relevance and impact of such models have been hampered by the circumstance that the gained insight could usually not be directly translated into improved patient care.

### SUMMARY OF THE INVENTION

It is an object of the present disclosure to provide a computer implemented method, computing device, system and computer program product for assessment of interconnection(s) between orthopaedic implant(s) and a bone tissue(s) of a specific patient that overcomes one or more of the disadvantages of the prior art.

According to the present disclosure, this object is addressed by the features of the independent claims. In addition, further advantageous embodiments follow from the dependent claims and the description.

In particular, it is an object of the present disclosure to provide a computer implemented method for assessment of interconnection(s) between orthopaedic implant(s) and a bone tissue(s) of a specific patient which allows the assessment of the risk that the interconnection between orthopaedic implant(s) and a bone tissue(s) becomes loose and which method is efficient in terms of computing power and hence requires a reduced amount of processing time.

In particular, the above object is addressed according to the present disclosure by a computer implemented method for assessment of interconnection(s) between orthopaedic implant(s) and a bone tissue(s) of a specific patient comprising the following method steps carried out by a computing device:
a. extracting location specific material properties of the bone tissue(s) from images of the bone tissue(s), in particular pre- and/or post-operative images;
b. generating a 3D-Finite Element Model comprising:
   first finite elements representing the orthopaedic implant interconnected to the bone tissue(s), and
   second finite elements representing at least part of the bone tissue(s) surrounding the orthopaedic implant;
c. applying the location-specific material properties to the second finite elements;
d. applying at least one external load to the one or more of the first finite elements;
e. determining internal stresses of the 3D-Finite Element Model;
f. outputting an assessment of the interconnection between orthopaedic implant(s) based on the determined internal stresses.

Inventors of the present disclosure recognized that known finite element-based methods for assessment of interconnections between orthopaedic implants and bone tissues failed to translate into improved patient care at least for the reason that such known finite element-based methods do not consider location-specific material properties of the bone tissue.

The present disclosure addresses this shortcoming of known finite element-based methods at least in that location specific material properties of the bone tissue(s) are extracted from pre- and/or post-operative images.

### Extracting Material Properties of the Bone Tissue(s)

Location specific material properties indicative of a quality of the bone tissue(s) are extracted from pre- and/or post-operative image(s) of the specific patient, in particular from pre-operative CT images. The extracted material properties are then assigned to volumetric elements of the 3D-Finite Element Model. The material properties comprise one or more of: density, elastic module, yield strength, ultimate strength, failure criteria (triaxial, cumulative), all properties being equal or different in tension and compression. According to an embodiment, bone density is calculated based on the Hounsfield units HU determined from the pre-operative CT images.

### Generating First Finite Elements Representing the Orthopaedic Implant

The computing device is configured to generate first finite elements representing the orthopaedic implant interconnected to the bone tissue(s).

According to embodiments disclosed herein, the first finite elements represent at least one out of the group of the following orthopaedic implant: fixation element, load transfer element, reinforcing screw, pedicle screw, reinforcing rod or a combination of at least two thereof.

According to embodiments, the first finite elements represent a combination of the orthopaedic implant, a reinforcing rod and at least one further orthopaedic implant arranged in the same or a different bone tissue(s). For example, first finite elements represent a reinforcement structure for interconnecting at least two vertebrae of a spine, the reinforcement structure being connected to the spine using pedicle screws.

According to embodiments disclosed herein, the location of the orthopaedic implant(s) are determined using an artificial intelligence-based model, supervised-learning and/or reinforcement learning being used to train the artificial intelligence-based model using a dataset comprising expert-identified ideal locations of similar orthopaedic implants.

Alternatively, or additionally, the location(s) of the orthopaedic implant(s) are determined semi-automatically or manually by a user based on a 3D-model of the bone tissue. According to an embodiment, the location of the orthopaedic implant(s) are indicated by a user using a pointing device, the pointing device being tracked by a tracking device and translated into data indicative of the location(s) of the orthopaedic implant(s) within the 3D-model of the bone tissue.

### Generating Second Finite Elements Representing at Least Part of the Bone Tissue(s) Surrounding the Orthopaedic Implant

The computing device is further configured to generate second finite elements using, if appropriate, the images of the bone tissue(s), the second finite elements representing at least part of the bone tissue(s) surrounding the orthopaedic implant, such as a cylinder arranged essentially coaxial to a bone screw. The second finite elements are generated based on pre- and/or post-operative image(s), such as radioscopic images, e.g. as Computed Tomography (CT) image(s) of the patient; low-dose Computed Tomography (CT) and/or MRI based pseudo-Computed Tomography images.

According to embodiments disclosed herein, the second finite elements are generated further using a database of 3D-models of similar bone tissues, in particular a database comprising information of 3D-models of bone tissues of people other than the specific patient.

In particular embodiments, the second finite elements are generated based on a CT image(s) of the patient by an artificial intelligence-based model, the artificial intelligence-based model having been trained using a dataset of CT images (of people other than the patient) along with corresponding vertebral level annotations.

According to embodiments, in specific cases of very osteoporotic bone tissue, wherein trabecular bone tissue does not provide significant support, the second finite elements comprise finite elements representing a cortical bone tissue area and finite elements representing trabecular bone tissue area. In order to conserve processing resources, according to embodiments, determining internal stresses of the 3D-Finite Element Model and/or determining a loading factor is/are limited to cortical bone tissue area.

### Applying the Location-Specific Material Properties to the Second Finite Elements

Having determined the second finite elements of the 3D-Finite Element Model, the computing device is configured to apply the location-specific material properties to the second finite elements.

Applying External Load onto the 3D-Finite Element Model

As a subsequent step, at least one external load is applied to the one or more of the first finite elements.

Inventors of the present disclosure further recognized that known finite element-based methods for assessment of interconnection(s) between orthopaedic implant(s) and a bone tissue(s) failed to translate into improved patient care at least for the reason that such known methods were limited to assessing axial pull-out strength(s) of the orthopaedic implant(s), which have a reduced correlation with the risk of loosening.

Embodiments of the present disclosure address this shortcoming by determining critical loading vector(s), wherein the external load is applied onto the 3D-Finite Element Model according to the critical loading vector(s). The critical loading vector(s) is/are determined specific to the particular bone tissue(s). Furthermore, the critical loading vector(s) is specific to the orthopaedic implant.

In order to achieve an even further accurate modelling of the orthopaedic implant(s) as implanted into the bone tissue(s) of the specific patient, according to further embodiments, at least a partial patient specific musculoskeletal model is created based on individual anatomy, alignment, and mass distribution. For example, pre- and/or post-operative images of the patient, in particular annotated radiographs of the erect posture can be used for the generation of an individualized musculoskeletal model. The individualized musculoskeletal model is used to determine a physiological load acting on the bone tissue, such as joints of the spine, during standing. The joint reaction forces, resulting from the physiological load acting on the bone tissue, are applied as load on the 3D-Finite Element Model.

### Determining Internal Stresses of the 3D-Finite Element Model

After applying the external load, the internal stresses of the 3D-Finite Element Model are calculated by the computing device. In particular, the internal stresses are determined as Von Mises stresses of the 3D-Finite Element Model.

In order to improve efficiency of the assessment of interconnection(s) between orthopaedic implant(s) and a bone tissue, according to embodiments, assessment area(s) of the 3D-Finite Element Model are defined, wherein the process determination of the internal stresses is limited to the finite elements within the assessment area(s). Alternatively, or additionally, the process of determining a loading factor is constrained to the finite element within the assessment area. In particular, the assessment area(s) is/are determined as geometrical envelope(s) of the orthopaedic implant(s), comprising at least those second finite elements, representing the bone tissue(s), adjacent to the orthopaedic implant as well as those first finite elements, representing the orthopaedic implant, adjacent to the second finite elements. In other words, the assessment area(s) are defined at the boundary between the orthopaedic implant(s) and the bone tissue based on specific boundary conditions. According to embodiments, the assessment area(s) is/are defined as geometrical envelope(s) of the orthopaedic implant(s) with a defined thickness, expressed as a physical distance and/or as a number of finite elements. Alternatively, the thickness of the geometrical envelope(s) of the orthopaedic implant(s) around the orthopaedic implant(s) is variable, the assessment area being extended to comprise adjacent finite elements of any finite element with a loading factor above an assessment threshold. Such embodiments are advantageous as critical areas are assessed while computing resources are conserved in less critical areas.

### Outputting an Assessment of the Interconnection Between Orthopaedic Implant(s) and the Bone Tissue(s)

Based on the determined internal stresses of the 3D-Finite Element Model, the computing device is configured to generate an assessment of the interconnection between the orthopaedic implant and the bone tissue(s). In particular, an assessment score is computed based on a comparison of loading factor(s) with loading factor threshold(s), the loading factor(s) having been calculated based on the determined internal stresses of the 3D-Finite Element Model.

The loading factor(s) is/are calculated as a function of a location-specific stress on the bone tissue(s) and a location-specific failure resistance of the bone tissue(s). According to particular embodiments, the loading factor(s) is/are calculated as a function of a Von Mises stress and a location-specific yield criterion. In particular, the loading factor is calculated as the relationship between the local internal stresses weighted by the local bone tissue(s) quality using the formula:

$$\begin{matrix}Loading Factor [ \% ] = \frac{{Stress}_{υ onMises}}{Failure Strength} \\ = mean ( \frac{{σ}_{υ onMises , i}}{{σ}_{Fail , i}} )\end{matrix}$$

where σ_{vonMises,i} and σ_{Fail,i} are the resulting Stress_{vonMises} and the ultimate failure strength of finite element i.

The assessment, as an assessment score, is then output/made available to a user. According to embodiments disclosed herein, the assessment is output using a display device communicatively connected to the computing device. Alternatively, or additionally, the assessment is output by the computing device as a data signal indicative of the assessment.

According to embodiments, a warning signal is generated if the loading factor(s) exceed(s) the loading factor threshold(s).

It is a further object of the present disclosure to provide a computing device for assessment of interconnection(s) between orthopaedic implant(s) and a bone tissue(s) of a specific patient which allows the assessment of the risk that the interconnection between orthopaedic implant(s) and a bone tissue(s) becomes loose and which method is efficient in terms of computing power and hence requires a reduced amount of processing time.

According to the present disclosure, this object is addressed by the features of the independent claims. In addition, further advantageous embodiments follow from the dependent claims and the description.

In particular, the above-identified object is further achieved by a computing device comprising a processing unit and a memory unit comprising instructions, which, when executed by the processing unit, cause the computing device to carry out the method according to one of the embodiment disclosed herein. The processing unit comprises any one or a combination of: a central processing unit CPU, a remote computing service, such as a cloud-based software as a service, and/or a dedicated hardware circuitry, such as a ASIC or FPGA. The memory unit comprises any one or a combination of: a volatile or non-volatile, optic, magnetic or semiconductor-based data storage device, and/or a cloud based datastore.

According to embodiments disclosed herein, the computing device comprises a data input interface communicatively connectable to an imaging device for capturing pre- and/or post-operative images of the patient. The data input interface, such as a wired (e.g. Ethernet, DVI, HDMI, VGA) and/or wireless data communication interface (e.g. 4G, 5G, Wifi, Bluetooth, UWB) is communicatively connectable to receive pre-operative imaging data therefrom. The data output interface such as a wired (e.g. Ethernet, DVI, HDMI, VGA) and/or wireless data communication interface (e.g. 4G, 5G, Wifi, Bluetooth, UWB) is configured to transmit at least part of assessment, respectively a visual representation of the assessment. Correspondingly, the computing device is configured for controlling the imaging device to capture the pre- and/or post-operative images of the bone tissue(s) comprising location-specific information about the material properties of the bone tissue(s) and for receiving the pre- and/or post-operative images from the imaging device.

It is a further object of the present disclosure to provide a system for assessment of interconnection(s) between orthopaedic implant(s) and a bone tissue(s) of a specific patient which allows the assessment of the risk that the interconnection between orthopaedic implant(s) and a bone tissue(s) becomes loose and which method is efficient in terms of computing power and hence requires a reduced amount of processing time.

According to the present disclosure, this object is addressed by the features of the independent claims. In addition, further advantageous embodiments follow from the dependent claims and the description.

In particular, the above-identified object is further achieved by a system comprising an imaging device and a computing device according to one of the embodiments disclosed herein. According to a particular embodiment, the imaging device is a CT imaging device configured to capture pre-operative CT image(s) of at least part of the bone tissue(s).

According to further embodiments, the system further comprises an output device, such as a computer screen, an augmented reality headset, or any device suitable to display a visual representation of the assessment of the interconnection between orthopaedic implant(s) and a bone tissue(s), the output device being communicatively connected to the computing device.

It is a further object of the present disclosure to provide a computer program product for assessment of interconnection(s) between orthopaedic implant(s) and a bone tissue(s) of a specific patient which allows the assessment of the risk that the interconnection between orthopaedic implant(s) and a bone tissue(s) becomes loose and which method is efficient in terms of computing power and hence requires a reduced amount of processing time.

According to the present disclosure, this object is addressed by the features of the independent claims. In addition, further advantageous embodiments follow from the dependent claims and the description.

In particular, the above-identified object is further achieved by a computer program product, comprising instructions, which, when carried out by a processing unit of a computing device, cause the computing device to carry out the method according to any one of the embodiments disclosed herein.

It is to be understood that both the foregoing general description and the following detailed description present embodiments, and are intended to provide an overview or framework for understanding the nature and character of the disclosure. The accompanying drawings are included to provide a further understanding, and are incorporated into and constitute a part of this specification. The drawings illustrate various embodiments, and together with the description serve to explain the principles and operation of the concepts disclosed.

### BRIEF DESCRIPTION OF SEVERAL VIEWS OF THE DRAWINGS

The herein described disclosure will be more fully understood from the detailed description given herein below and the accompanying drawings which should not be considered limiting to the disclosure described in the appended claims.

FIG. 1 shows a flowchart illustrating the steps according to a particular embodiment of the method for assessment of interconnection(s) between orthopaedic implant(s) and a bone tissue;

FIG. 2 shows an abstract illustration of a bone tissue and an orthopaedic implant attached thereto, showing the assessment area around the orthopaedic implant as well as the external load according to a critical loading vector;

FIG. 3 shows abstract illustrations of a bone tissue and a plurality of orthopaedic implants attached thereto form several perspectives;

FIG. 4 shows abstract illustrations of a 3D-Finite Element Model of the bone tissue and the plurality of orthopaedic implants of FIG. 3 from several perspectives, illustrating the assessment areas;

FIG. 5 shows abstract illustrations of an embodiment, wherein several bone tissues are assessed, each bone tissue having a plurality of orthopaedic implants attached thereto, form several perspectives;

FIG. 6 shows abstract illustrations of a 3D-Finite Element Model of the bone tissue(s) and the plurality of orthopaedic implants of FIG. 5 from several perspectives, illustrating the assessment areas;

FIG. 7 shows a highly schematic perspective view of visual representations of a spinal 3D-model and of a reinforcement 3D-model of a reinforcement structure;

FIG. 8 shows an illustration of extracting material properties indicative of a bone tissue(s) quality of vertebrae from pre-operative CT images of a spine;

FIG. 9 shows a sequence of illustrations of applying a spinal alignment onto a patient's spine as a particular case of a bone tissue, generating a spinal 3D-model, generating a musculoskeletal model respectively of a 3D-Finite Element Model representing the musculoskeletal model as well as a reinforcement structure as a particular case of orthopaedic implant;

FIG. 10 shows a visual representation of a 3D-Finite Element Model according to the present disclosure, illustrating the load applied onto the 3D-Finite Element Model in a caudo-cranial direction;

FIG. 11 shows a partial visual representation of a 3D-Finite Element Model according to the present disclosure, illustrating the load on a single pedicle screw;

FIG. 12 shows a visual representation of a 3D-Finite Element Model of a specific use case related to pedicle screws as orthopaedic implants according to the prior art, for calculating a pull-out strength of a single pedicle screw for assessing a pedicle screw, the load being applied in an axial direction (of the pedicle screw);

FIG. 13 shows a partial visual representation of a 3D-Finite Element Model according to the present disclosure, illustrating applying the load onto the 3D-Finite Element Model in a caudo-cranial direction;

FIG. 14 shows a visual representation of a 3D-Finite Element Model according to the present disclosure, illustrating the local internal stresses in the bone tissue(s) structure of a pedicle around a pedicle screw as an example of an orthopaedic implant; and

FIG. 15 shows a highly schematic block diagram of a system for supporting selection between different types of pedicle screws according to the present disclosure.

### DETAILED DESCRIPTION OF THE INVENTION

Reference will now be made in detail to certain embodiments, examples of which are illustrated in the accompanying drawings, in which some, but not all features are shown.

Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

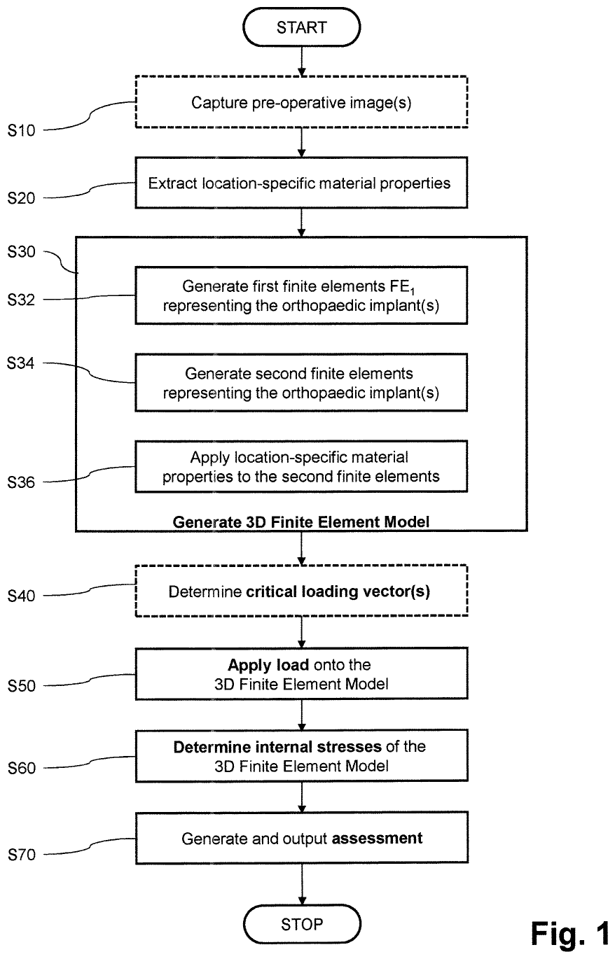

FIG. 1 shows a flowchart illustrating the steps according to a particular embodiment of the method for assessment of interconnection(s) between orthopaedic implant(s) 21₁₋ₘ and a bone tissue(s) 201, 201₁₋ₙ.

In a preparatory step S10, radioscopic pre- and/or post-operative image(s), such as CT image(s), of the patient are captured using an imaging device 50, such as a CT imaging device, of at least part of the bone tissue(s) 201, 201₁₋ₙ.

In step S20, location-specific material properties indicative of a quality of the bone tissue(s) 201, 201₁₋ₙ are extracted from pre- and/or post-operative image(s) of the bone tissue(s) 201, 201₁₋ₙ of the specific patient, in particular from pre-operative CT images of the bone tissue(s) 201, 201₁₋ₙ. The material properties comprise one or more of: stiffness; bone density and/or bone strength. According to an embodiment, bone density is calculated based on the Hounsfield units HU determined from the pre-operative CT images.

In step S30, a 3D-Finite Element Model FE is generated by the computing device 10, in a series of substeps S32, S34 and S36.

In substep S32, first finite elements FE_{I} representing the orthopaedic implant 21₁₋ₙ interconnected to the bone tissue(s) 201, 201₁₋ₙ are generated. The location of the orthopaedic implant(s) 21₁₋ₘ within the bone tissue(s) 201, 201₁₋ₙ is/are determined using an artificial intelligence-based model, supervised-learning and/or reinforcement learning being used to train the artificial intelligence-based model using a dataset comprising expert-identified ideal locations of similar orthopaedic implants 21₁₋ₙ. Alternatively, or additionally, the location(s) of the orthopaedic implant(s) 21₁₋ₘ are determined semi-automatically or manually by a user based on a 3D-model of the bone tissue(s) 201, 201₁₋ₙ. Alternatively, or additionally, the location of the orthopaedic implant(s) 21₁₋ₙ are indicated by a user using a pointing device, the pointing device being tracked by a tracking device and translated into data indicative of the location(s) of the orthopaedic implant(s) 21₁₋ₘ within the 3D-model of the bone tissue(s) 201, 201₁₋ₙ.

Depending on the orthopaedic implant 21₁₋ₘ, location-specific physical and/or mechanical properties of the orthopaedic implant(s) 21₁₋ₘ are applied to the first finite elements FE_{I}. Alternatively, whenever the mechanical properties such as strength, toughness, hardness of the orthopaedic implant(s) 21₁₋ₘ can be assumed to be several orders of magnitude above those of the bone tissue(s) 201, 201₁₋ₙ, location-specific physical and/or mechanical properties of the orthopaedic implant(s) 21₁₋ₘ need not be considered as the assessment of the interconnection between the orthopaedic implant(s) 21₁₋ₘ and the bone tissue(s) 201, 201₁₋ₙ will be determined by the quality of the bone tissue(s) 201, 201₁₋ₙ.

In a further substep S34, second finite elements FE_{II} representing the bone tissue(s) 201, 201₁₋ₙ are generated using the pre- and/or post-operative images of the bone tissue(s) 201, 201₁₋ₙ. In order to conserve computing resources, the generation of the second finite elements FE_{II} may be limited to an area surrounding the orthopaedic implant 21₁₋ₘ. The second finite elements FE_{II} are generated based on a CT image(s) of the patient by an artificial intelligence-based model, the artificial intelligence-based model having been trained using a dataset of CT images (of people other than the patient) along with corresponding vertebral level annotations.

Subsequent to generating the second finite elements FE_{II}, in a substep S36, the location-specific material properties are applied to the second finite elements FE_{II}.

In a step S40, critical loading vector(s) L_{cr} is/are determined specific to the particular bone tissue(s) 201, 201₁₋ₙ and specific to the orthopaedic implant 21₁₋ₘ. The critical loading vector(s) L_{cr} is/are determined as a vector of the highest expected load onto the interconnection between orthopaedic implant(s) 21₁₋ₘ and a bone tissue(s) 201, 201₁₋ₙ. In particular, the critical loading vector(s) La is/are dependent not only on the characteristics of the orthopaedic implant(s) 21₁₋ₘ but also on their special orientation within the bone tissue(s) 201, 201₁₋ₙ. Depending on the specific use case, the critical loading vector(s) correspond to a direction of highest momentarily load (such as a torsional load due to a movement of a patient) or a highest average load over a time span (such as the gravitational load acting over extended periods of time).

In order to achieve an even further accurate modelling of the orthopaedic implant(s) 21₁₋ₘ as implanted into the bone tissue(s) 201, 201₁₋ₙ of the specific patient, at least a partial patient specific musculoskeletal model M_{Musc} is created based on individual anatomy, alignment, and mass distribution. For example, pre- and/or post-operative images of the patient, in particular annotated radiographs of the erect posture can be used for the generation of an individualized musculoskeletal model M_{Musc}. The individualized musculoskeletal model M_{Musc} is used to determine a physiological load acting on the bone tissue(s) 201, 201₁₋ₙ, such as joints of the spine, during standing. The joint reaction forces, resulting from the physiological load acting on the bone tissue(s) 201, 201₁₋ₙ, are applied as load on the 3D-Finite Element Model.

Thereafter, in step S50, at least one external load Lₑₓₜ is applied to the one or more of the first finite elements FE_{I} according to the critical loading vector(s). Depending on the specific orthopaedic implant(s) 21₁₋ₘ, the external load Lₑₓₜ is applied to a specific group of the first finite elements FE_{I} representing a fixation, support area of the orthopaedic implant(s) 21₁₋ₘ, such as the interconnection between a reinforcement structure 30 (for interconnecting multiple vertebrae 200₁₋ₙ of a spine 202) and orthopaedic implant(s) 21₁₋ₘ attaching the reinforcement structure 30 to the vertebrae 200₁₋ₙ.

In step S60, internal stresses S of the 3D-Finite Element Model FE are calculated within assessment area(s) FE_{eval.1-m}. The assessment area(s) FE_{eval.1-m} of the 3D-Finite Element Model FE is/are defined as geometrical envelope(s) of the orthopaedic implant(s) 21₁₋ₘ, comprising at least the second finite elements FE_{II} representing the bone tissue(s) 201, 201₁₋ₙ adjacent to the orthopaedic implant 21₁₋ₙ as well as the first finite elements FE_{I} adjacent to the second finite elements FE₂.

The assessment area(s) FE_{eval.1-m} is/are defined as geometrical envelope(s) of the orthopaedic implant(s) 21₁₋ₘ with a defined thickness, expressed as a physical distance and/or as a number of finite elements. Alternatively, the thickness of the geometrical envelope(s) of the orthopaedic implant(s) 21₁₋ₘ is variable, the assessment area FE_{eval.1-m} being gradually extended to comprise adjacent finite elements of any finite element with a calculated loading factor above an assessment threshold.

Finally, in step S70, based on the determined internal stresses S of the 3D-Finite Element Model FE, an assessment of the interconnection between the orthopaedic implant 21₁₋ₘ and the bone tissue(s) 201, 201₁₋ₙ is generated. In particular, an assessment score is computed based on a comparison of loading factor(s) with loading factor threshold(s), the loading factor(s) having been calculated based on the determined internal stresses S of the 3D-Finite Element Model FE.

The loading factor(s) is/are calculated as a function of a location-specific stress on the bone tissue(s) 201, 201₁₋ₙ and a location-specific failure resistance of the bone tissue(s) 201, 201₁₋ₙ. According to particular embodiments, the loading factor(s) is/are calculated as a function of a Von Mises stress and a location-specific yield criterion. In particular, the loading factor is calculated as the relationship between the local internal stresses S weighted by the local bone tissue(s) 201, 201₁₋ₙ quality using the formula:

$$\begin{matrix}Loading Factor [ \% ] = \frac{{Stress}_{υ onMises}}{Failure Strength} \\ = mean ( \frac{{σ}_{υ on Mises , i}}{{σ}_{Fail , i}} )\end{matrix}$$

where σ_{vonMises,i} and σ_{Fail,i} are the resulting Stress_{VonMises} and the ultimate failure strength of finite element i.

The assessment, as an assessment score, is then output/made available to a user. According to embodiments disclosed herein, the assessment is output using a display 60 device communicatively connected to the computing device 10. Alternatively, or additionally, the assessment is output by the computing device 10 as a data signal indicative of the assessment.

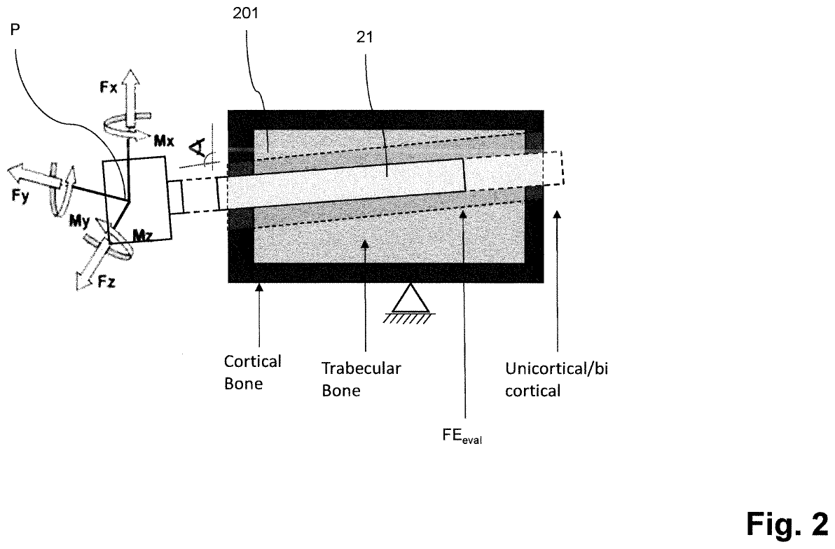

FIG. 2 shows an abstract illustration of a bone tissue 201 and an orthopaedic implant 21 attached thereto, showing the assessment area FEₑᵥₐₗ around the orthopaedic implant 21 as well as the external load Lₑₓₜ according to a critical loading vector. As illustrated on FIG. 2, the critical loading vector may be determined in the Cartesian coordinate system as a set of normal forces Fₓ, F_{y} and F_{z} (tension or compression) and a set of torsional forces Mₓ, M_{y} and M_{z} in the x, y and z coordinates the forces being applied into a finite element representing a loading point P of the orthopaedic implant 21.

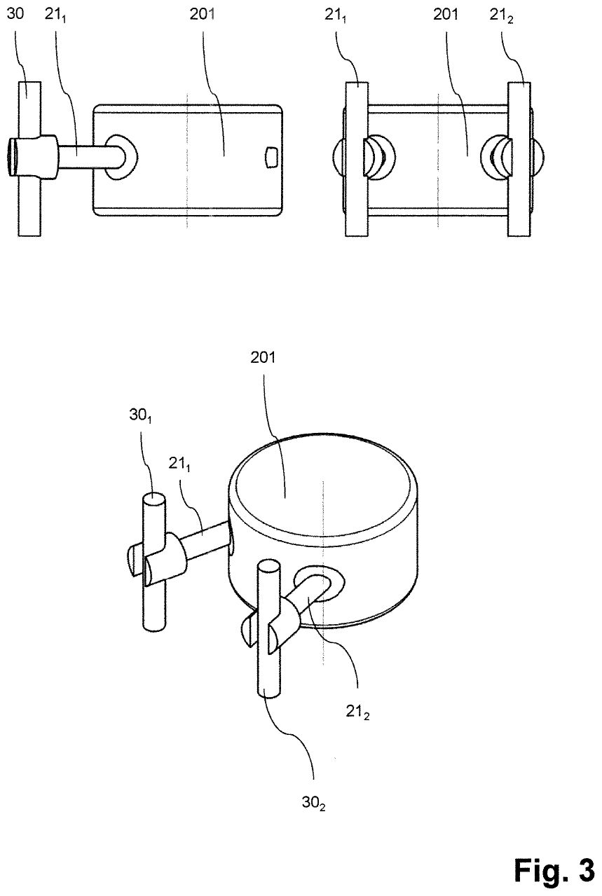

FIG. 3 shows a bone tissue 201 and a plurality of orthopaedic implants 21₁₋ₘ attached thereto form several perspectives. As illustrated, the orthopaedic implants 21₁₋ₘ are attached to a reinforcement structure 30, 30₁₋ₘ.

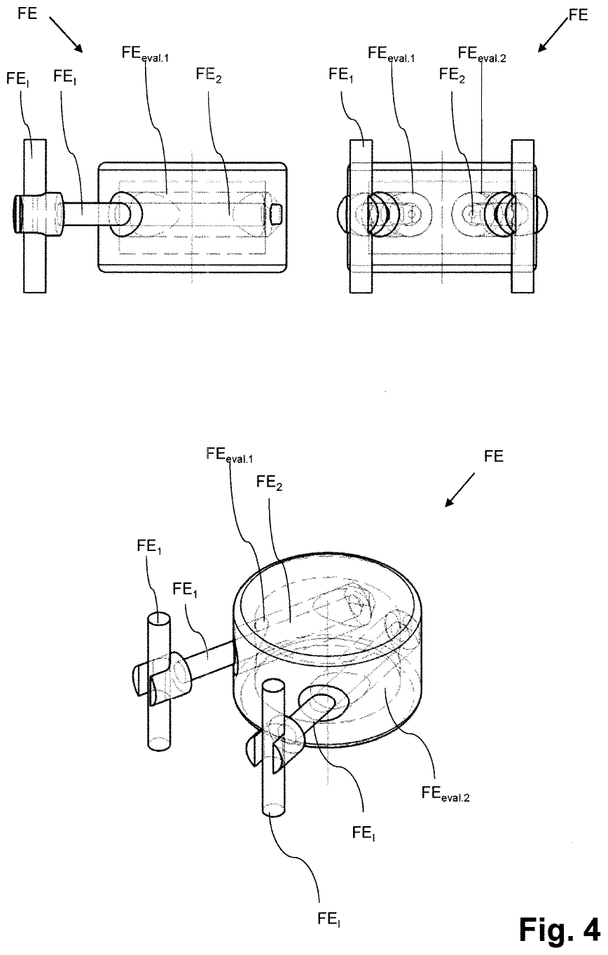

FIG. 4 shows the bone tissue 201 and the plurality of orthopaedic implants 21₁₋ₘ of FIG. 3 from several perspectives, illustrating the assessment areas FE_{eval.1-m}. As shown, the assessment areas FE_{eval.1-m} are defined as cylindrical areas within the bone tissue 201 around the orthopaedic implants 21₁₋ₘ.

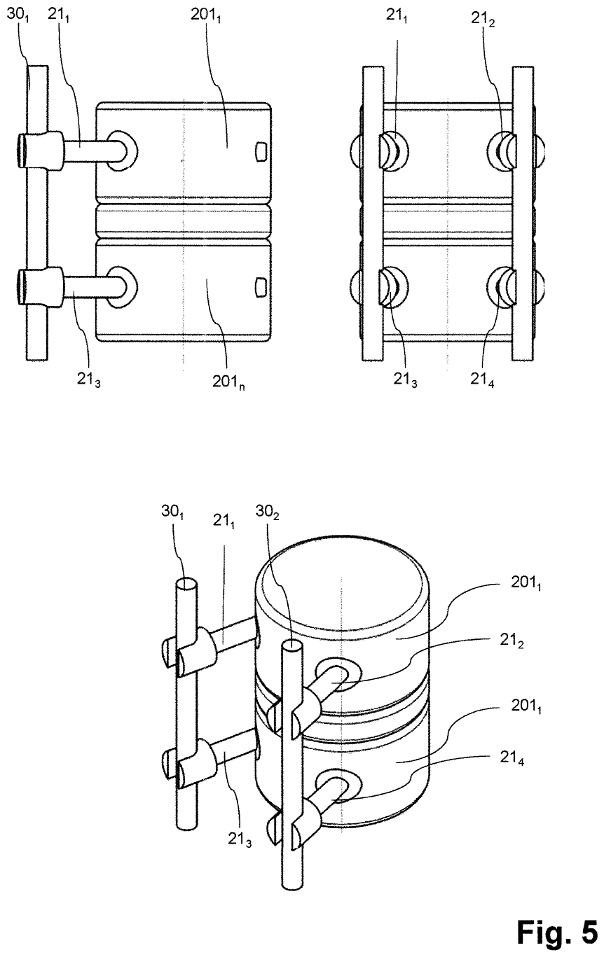

FIG. 5 shows an embodiment, wherein several bone tissues 201₁₋ₙ are assessed, each bone tissue 201₁₋ₙ having a plurality of orthopaedic implants 21₁₋ₘ attached thereto, form several perspectives. As illustrated, the orthopaedic implants 21₁₋ₘ are attached to reinforcement structures 30₁₋ₘ.

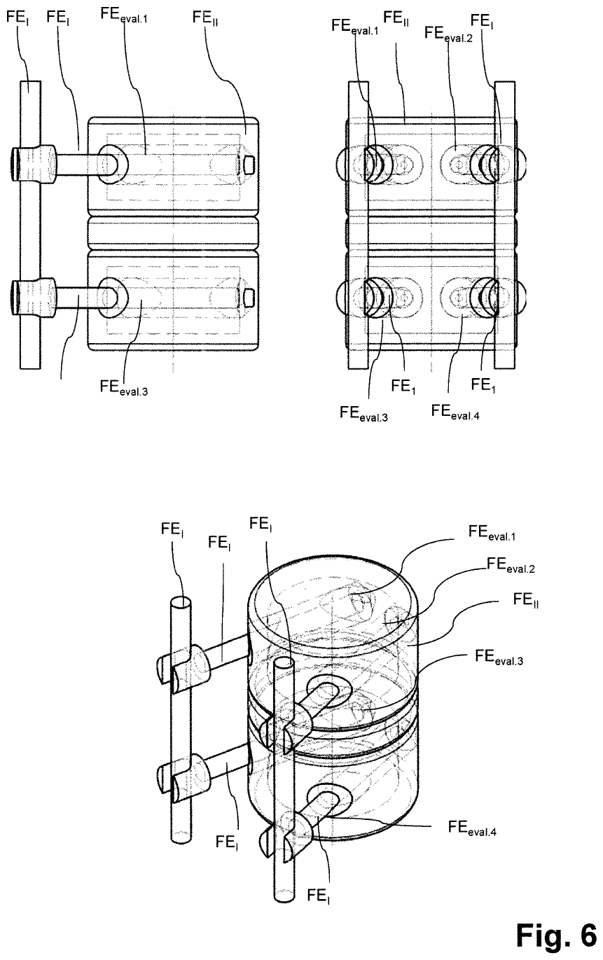

FIG. 6 shows the bone tissue(s) 201₁₋ₙ and the plurality of orthopaedic implants 21₁₋ₘ of FIG. 5 from several perspectives, illustrating the assessment areas FE_{eval.1-m}. As shown, the assessment areas FE_{eval.1-m} are defined as cylindrical areas within the bone tissue(s) 201₁₋ₙ around the orthopaedic implants 21₁₋ₘ.

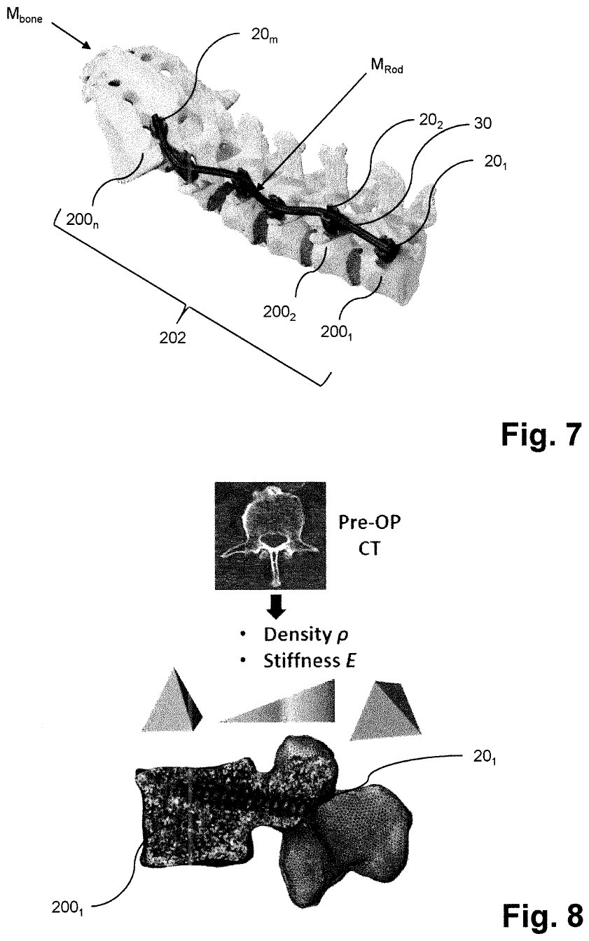

The present disclosure shall be described in the following with reference to FIGS. 7 to 14 depicting a specific use case for the assessment of an interconnection between pedicle screws 20₁₋ₘ as orthopaedic implants and pedicles 200₁₋ₙ as bone tissues.

FIG. 7 shows a highly schematic perspective view of a visual representation of a spinal 3D-model M_{bone} (shown in light grey) and of a reinforcement 3D-model M_{Rod} of a reinforcement structure 30 (shown in dark grey). The spinal 3D-model M_{bone} captures multiple vertebrae 200₁₋ₙ of a patient's spine 202. As illustrated, intervertebral disc(s) are also captured by the spinal 3D-model M_{bone}. Overlaid onto the spinal 3D-model M_{bone}, FIG. 7 also depicts the reinforcement 3D-model M_{Rod} of a reinforcement structure 30 for interconnecting multiple vertebrae 200₁₋ₙ of the spine 202. In the illustrated embodiments, the reinforcement structure 30 is a reinforcement rod for being attached to U-shaped openings (so-called tulips) of the pedicle screw heads as an example of orthopaedic implants 21₁₋ₘ. The reinforcement structure 30 is specific to the positioning of the pedicle screws 20₁₋ₘ within the patient's spine, being shaped according to the spatial positions of a plurality of chirurgical implants (pedicle screws) attached to a patient.

The pedicle screws 20₁₋ₘ are arranged within the vertebrae 200₁₋ₙ of as described in PCT/EP2021/085554, the disclosure of which is hereby incorporated by reference.

FIG. 8 illustrates how material properties indicative of a bone quality of the vertebrae 200₁₋ₙ, such as density and stiffness, are extracted from pre-operative CT images of the spine 202. Bone density of the vertebrae 200₁₋ₙ is calculated based on the Hounsfield units HU determined from the pre-operative CT images of the spine 202. In order to improve accuracy, variable tube voltage used for acquisition of the pre-operative CT images may be accounted for by correcting the HU values.

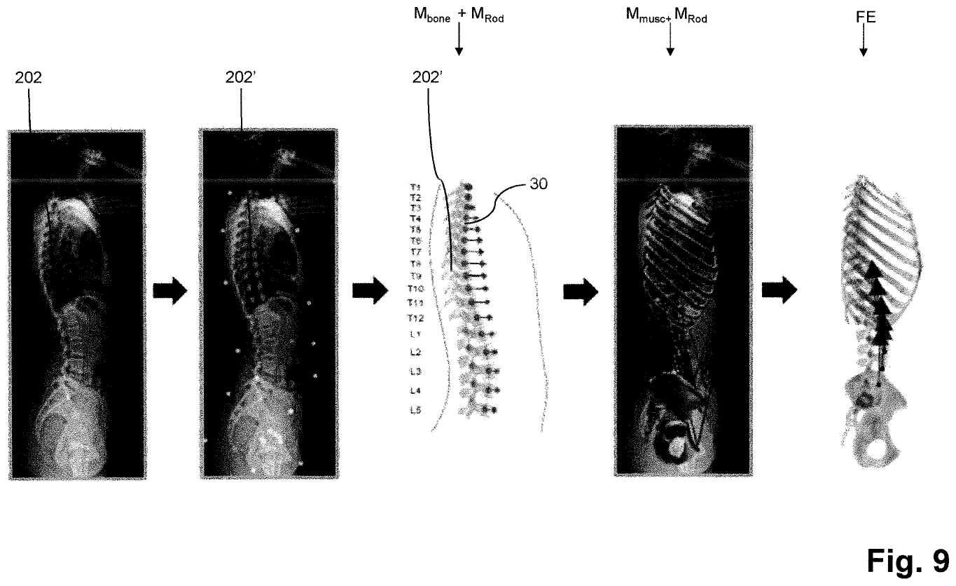

FIG. 9 depicts a sequence of illustrations of applying a spinal alignment onto a patient's spine 202, generating a spinal 3D-model M_{bone}, generating a musculoskeletal model M_{Musc}, respectively of a 3D-Finite Element Model FE representing the musculoskeletal model M_{Musc} as well as the reinforcement structure 30.

The leftmost illustration of FIG. 9 depicts a preoperative radiographic image of a patient's spine 202 as captured by an imaging device 50. The second illustration from the left of FIG. 9 depicts an aligned spine 202′ after a spinal alignment process has been applied onto the spine 202. The spinal alignment process is applied such that the anatomy of the spine is (at least partially) corrected. The illustration in the middle of FIG. 9 depicts a visual representation of the spinal 3D-model M_{bone}, generated after the spinal alignment overlaid onto a visual representation of the reinforcement 3D-model M_{Rod} generated on the basis of the spinal 3D-model M_{bone}. The second illustration from the right on FIG. 9 depicts a visual representation of a musculoskeletal model M_{Musc} generated based on individual anatomy, alignment, and mass distribution as determined based on preoperative images of the patient.

Finally, the rightmost illustration of FIG. 9 depicts a visual representation of the 3D-Finite Element Model FE representing the musculoskeletal model M_{Musc}, the multiple vertebrae 200₁₋ₙ as captured by the spinal 3D-model M_{bone} and further representing the reinforcement structure 30 attached to the vertebrae 200₁₋ₙ using pedicle screws 20₁₋ₘ as an example of orthopaedic implants 21₁₋ₘ.

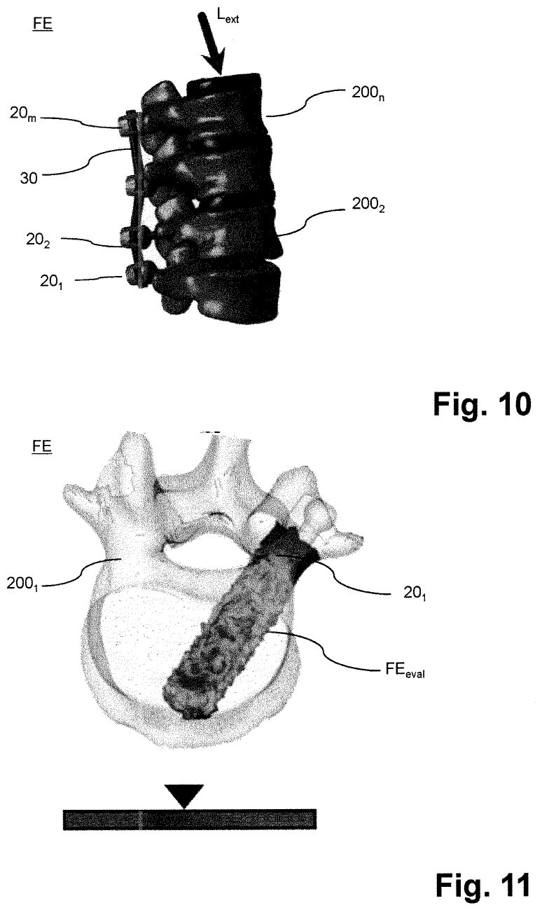

FIG. 10 shows a visual representation of the 3D-Finite Element Model FE according to the present disclosure, illustrating the load Lₑₓₜ applied onto the 3D-Finite Element Model FE in a caudo-cranial direction C-C. The 3D-Finite Element Model FE is generated corresponding to the spinal 3D-model M_{bone} and the reinforcement 3D-model M_{Rod}. Therefore, the 3D-Finite Element Model FE represents the vertebrae 200₁₋ₙ as captured by the spinal 3D-model M_{bone}; and the reinforcement structure 30 attached to the vertebrae 200₁₋ₙ using at least the pedicle screws 20₁₋ₘ as an example of orthopaedic implants 21₁₋ₘ.

As illustrated with a thick, downward pointing arrow, the load Lₑₓₜ is applied onto the 3D-Finite Element Model FE according to a critical load vector L_{cr}, specifically a caudo-cranial direction C-C, for example on the uppermost vertebra 200ₙ. In other words, the load Lₑₓₜ is applied as if pulling the vertebra 200ₙ down with respect to the reinforcement structure 30 attached to the spine 202 using pedicle screws 20₁₋ₘ as an example of orthopaedic implants 21₁₋ₘ.

In order to calculate the loading factor, a force at peak velocity, mean HU values, von Mises Stress Stress_{VonMises} values are computed. The loading factor is calculated as the relationship between the local internal stresses weighted by the local bone tissue(s) 201, 201₁₋ₙ quality using the formula:

$$\begin{matrix}Loading Factor [ \% ] = \frac{{Stress}_{υ onMises}}{Failure Strength} \\ = mean ( \frac{{σ}_{υ on Mises , i}}{{σ}_{Fail , i}} )\end{matrix}$$

where σ_{vonMises,i} and σ_{Fail,i} are the resulting Stress_{VonMises} and the ultimate failure strength of finite element i, respectively. FIG. 10 shows this for orthopaedic implants 21₁₋ₘ for the example of a pedicle screw 20ᵢ.

For cases where a pair of pedicle screws is to be attached to the left and the right pedicle(s), a mean value between the left and the right pedicle is considered for determining the loading factor. This way, for every assessed parameter there was one data point per vertebra 200₁₋ₙ.

FIG. 11 shows a partial visual representation of the 3D-Finite Element Model FE according to the present disclosure, illustrating the calculated load on a single pedicle screw 201, representative of an orthopaedic implant 21₁, implanted into a vertebra 200₁. As shown on FIG. 11, an assessment area FEₑᵥₐₗ is defined around the pedicle screw 20₁.

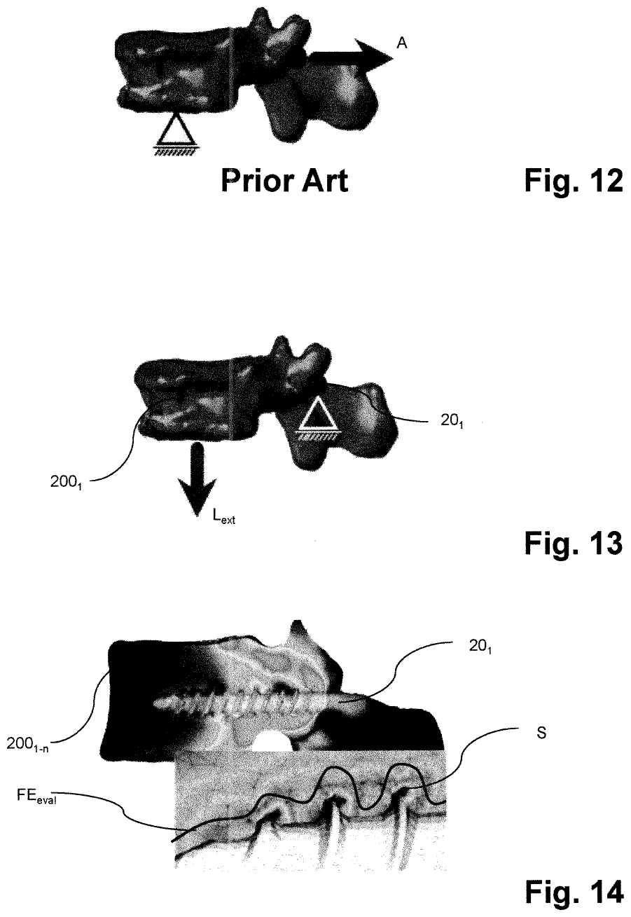

FIG. 12 shows visual representation of a 3D-Finite Element Model according to the prior art for calculating a pull-out strength of a single pedicle screw for assessing a pedicle screw. However, it was recognized that prior art 3D-Finite Element Models wherein load is applied onto the pedicle screws in an axial direction (see bold arrow on FIG. 12) do not yield meaningful assessment of the risk of pedicle screw loosening in patients.

As illustrated on FIG. 13, in contrast to the 3D-Finite Element Model according to the prior art shown on FIG. 12, the 3D-Finite Element Model FE according to the present disclosure is loaded Lₑₓₜ in accordance with a critical load vector, in the use case illustrated on FIGS. 7 to 14, a caudo-cranial load. Loading the 3D-Finite Element Model FE, according to the present disclosure, in accordance with a critical load vector, in the use case illustrated on FIGS. 7 to 14, is advantageous as it allows a much more relevant, real-life assessment of the behaviour of the pedicle screws 20₁ in a patient, wherein the most significant stress applied onto the vertebrae 200₁ is the gravitational force in an erect posture of the spine. The vertical gravitational force most commonly translates to a stress onto the pedicle screws 20₁ which significantly deviates from an axial direction (with respect to a usually longitudinal axis of the pedicle screws), due to anatomical constraints on the orientation of the pedicle screws implanted into the vertebrae 200₁₋ₙ.

FIG. 14 shows a visual representation of the 3D-Finite Element Model FE according to the present disclosure, illustrating the local internal stresses S in the bone structure of a pedicle 200₁ around a pedicle screw 20₁, the pedicle screw 20₁ representing a form of orthopaedic implant 21₁. As illustrated on the magnified view on the bottom right side of FIG. 14, significant stresses commonly occur around the thread of the pedicle screw 20₁. As shown on FIG. 14, an assessment area FEₑᵥₐₗ is defined as a geometrical envelope of the pedicle screw 20₁, the assessment of an interconnection between the pedicle screws 20₁₋ₘ and the vertebrae 200₁₋ₙ is based on the determination of the internal stresses S and loading factors determined within the assessment area FEₑᵥₐₗ.

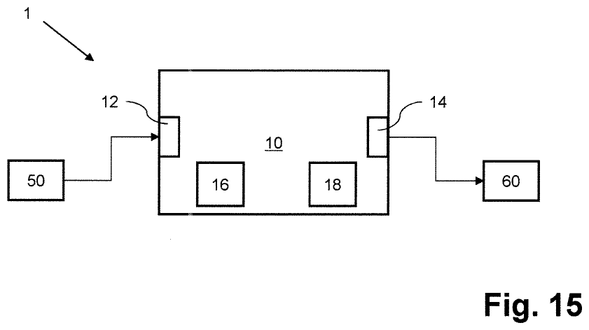

FIG. 15 shows a highly schematic block diagram of a system 1 for the assessment of interconnection(s) between orthopaedic implant(s) (21₁₋ₙ) and bone tissue(s) (201, 201₁₋ₙ) according to the present disclosure. The system 1 comprises an imaging device 50 and a computing device 10 comprising a processing unit 16 and a memory unit 18 comprising instructions, which, when executed by the processing unit 16, cause the computing device 10 to carry out the method according to one of the embodiment disclosed herein. The processing unit 16 comprises any one or a combination of: a central processing unit CPU, a remote computing service, such as a cloud-based software as a service, and/or a dedicated hardware circuitry, such as an ASIC or FPGA. The memory unit 14 comprises any one or a combination of: a volatile or non-volatile, optic, magnetic or semiconductor-based data storage device, and/or a cloud based datastore.

The imaging device 50 may be a CT imaging device configured to capture pre-operative CT image(s) of at least part of the spine 202 capturing at least two vertebrae 200₁₋ₙ of the spine 202, the imaging device 50 being communicatively connected to the computing device 10 via a data input interface 12. The data input interface 12 is any one of or a combination of a wired (e.g. Ethernet, DVI, HDMI, VGA) and/or wireless data communication interface (e.g. 4G, 5G, Wifi, Bluetooth, UWB).

The system 1 further comprises an output device 60, such as a computer screen, an augmented reality headset, or any device suitable to display a visual representation of the assessment of interconnection(s) between orthopaedic implant(s) 21₁₋ₘ and bone tissue(s) 20₁, 201₁₋ₙ, the output device 60 being communicatively connected to the computing device 10 via a data output interface 14. The data output interface 14 is any one of or a combination of a wired (e.g. Ethernet, DVI, HDMI, VGA) and/or wireless data communication interface (e.g. 4G, 5G, Wifi, Bluetooth, UWB) is configured to transmit at least part of the assessment, respectively a visual representation of the assessment.

1. A computer implemented method for assessment of interconnection(s) between orthopaedic implant(s) (21₁₋ₙ) and bone tissue(s) (201, 201₁₋ₙ) of a specific patient comprising the following method steps carried out by a computing device (10):
a. extracting location specific material properties of the bone tissue(s) (20₁, 201₁₋ₙ) from pre- and/or post-operative images of the bone tissue(s) (201, 201₁₋ₙ);
b. generating a 3D-Finite Element Model (FE) comprising:
i. first finite elements (FE_{I}) representing the orthopaedic implant (21₁₋ₐ) interconnected to the bone tissue(s) (201, 201₁₋ₙ), and
ii. second finite elements (FE_{II}) representing at least part of the bone tissue(s) (201, 201₁₋ₙ) surrounding the orthopaedic implant (21₁₋ₙ);
c. applying the location-specific material properties to the second finite elements (FE_{II});
d. applying at least one external load (Lₑₓₜ) to the one or more of the first finite elements (FE_{I});
e. determining internal stresses (S) of the 3D-Finite Element Model (FE);
f. outputting an assessment of the interconnection between the orthopaedic implant(s)(21₁₋ₙ) and the bone tissue(s) (201, 201₁₋ₙ) based on the determined internal stresses (S).

2. The computer implemented method according to claim 1, further comprising determining loading factor(s) based on the determined internal stresses (S) of the 3D-Finite Element Model (FE), wherein outputting an assessment of the interconnection between the orthopaedic implant(s) (21₁₋ₙ) and the bone tissue(s) (201, 201₁₋ₙ) comprises outputting an assessment score based on a comparison of the loading factor(s) with loading factor threshold(s).

3. The computer implemented method according to claim 2, further comprising determining assessment area(s) (FE_{eval.1-n}) of the 3D-Finite Element Model (FE) as a geometrical envelope of the orthopaedic implant (21₁₋ₙ), comprising at least the second finite elements (FE_{II}) adjacent to the orthopaedic implant (21₁₋ₙ) and the first finite elements (FE_{I}) adjacent to the second finite elements (FE_{II}), wherein:
a. determining the internal stresses (S) of the 3D-Finite Element Model (FE) comprises determining the internal stresses (S) for each finite element within the assessment area(s) (FE_{eval.1-n}); and/or
b. determining a loading factor comprises determining location-specific loading factor(s) for each finite element within the assessment area (FE_{eval.1-n}).

4. The computer implemented method according to claim 1, further comprising determining one or more critical loading vector(s) corresponding to the bone tissue(s) (201, 201₁₋ₙ) and/or corresponding to the orthopaedic implant (21₁₋ₙ), wherein the external load (Lₑₓₜ) is applied onto the 3D-Finite Element Model (FE) according to the critical loading vector(s).

5. The computer implemented method according to claim 4, further comprising:
a. creating at least a partial patient-specific musculoskeletal model (M_{Musc}) comprising the bone tissue(s) (201, 201₁₋ₙ); and
b. determining the critical loading vector(s) using the patient-specific musculoskeletal model (M_{Musc}).

6. The computer implemented method according to claim 4, wherein the critical loading vector(s) comprise a caudo-cranial direction (C-C) and/or a torsional direction.

7. The computer implemented method according to claim 1, wherein the loading factor(s) is/are calculated as a function of a location-specific stress on the bone tissue(s) (201, 201₁₋ₙ) and a location-specific failure resistance of the bone tissue(s) (201, 201₁₋ₙ).

8. The computer implemented method according to claim 7, wherein the loading factor(s) is/are calculated as a function of a Von Mises stress and a location-specific yield criterion.

9. The computer implemented method according to claim 1, wherein the second finite elements (FE) comprise finite elements representing a cortical bone tissue area and finite elements representing trabecular bone tissue area.

10. The computer implemented method according to claim 1, wherein
a. determining internal stresses (S) of the 3D-Finite Element Model (FE); and/or
b. determining a loading factor is/are limited to cortical bone tissue area.

11. The computer implemented method according to claim 1, wherein the first finite elements (FE_{I}) represent at least one out of the group of the following orthopaedic implants (21₁₋ₙ): a fixation element, a load transfer element, a reinforcing screw, a pedicle screw, a reinforcing rod or a combination thereof.

12. The computer implemented method according to claim 1, wherein the second finite elements (FE_{II}) represent one or more vertebrae (200₁₋ₙ) or pedicle(s) of vertebrae and wherein the first finite elements (FE) represent pedicle screw(s) (20₁₋ₘ) arranged within the vertebrae (200₁₋ₙ).

13. The computer implemented method according to claim 1, wherein the first finite elements (FE_{I}) represent a combination of the orthopaedic implant (21₁₋ₙ), a reinforcing rod and a least one further orthopaedic implant (21₁₋ₙ) arranged in the same or a different bone tissue(s) (201, 201₁₋ₙ).

14. The computer implemented method according to claim 1, further comprising:
a. controlling an imaging device to capture the pre- and/or post-operative images of the bone tissue(s) (201, 201₁₋ₙ) comprising location-specific information about the material properties of the bone tissue(s) (201, 201₁₋ₙ);
b. receiving the images of the bone tissue(s) (201, 201₁₋ₙ) from the imaging device.

15. A computing device (10) comprising a processing unit (16) and a memory unit (18) comprising instructions, which, when executed by the processing unit (16) cause the computing device (10) to carry out the method according to claim 1.

16. A system comprising:
a. an imaging device (50), in particular a CT imaging device, communicatively connected to the computing device (10), the imaging device (50) being configured to capture pre- and/or post-operative image(s) of at least part of the bone tissue(s) (201, 201₁₋ₙ); and
b. a computing device (10) according to claim 15.

17. A computer program product, comprising instructions, which, when carried out by a processing unit (16) of a computing device (10), cause the computing device (10) to carry out the method according to claim 1.
